(19) United States
(12) Patent Application Publication
(10) Pub. No.: US 2021/0351624 A1
(43) Pub. Date: Nov. 11, 2021

(54) LOCAL SUPPLY VOLTAGE REGULATION OF A RECHARGEABLE MEDICAL IMPLANT VIA RESONANCE TUNING

(71) Applicant: BIOTRONIK SE & Co. KG, Berlin (DE)

(72) Inventor: Gregory Jay Delmain, Minnetrista, MN (US)

(21) Appl. No.: 17/307,131

(22) Filed: May 4, 2021

Related U.S. Application Data
(60) Provisional application No. 63/020,565, filed on May 6, 2020.

(51) Int. Cl. H02J 50/12; H02J 7/02; H03K 17/687; A61N 1/378; A61N 1/36

(52) U.S. Cl. CPC H02J 50/12; H02J 7/02; H03K 17/6871; A61N 1/36082; A61N 1/36053; A61N 1/36062; A61N 1/3787

(57) ABSTRACT

An implantable medical device (1) includes a resonant circuit, a switch (Si), and a control circuit (CC). The resonant circuit includes an inductive charging coil (L1), a first capacitor and (C1′), and a second capacitor (C2′). The inductive charging coil (L1) is electrically connected to the first capacitor (C1′). The inductive charging coil (L1) is electrically connected to the second capacitor (C2′) when the switch (S1) is closed and electrically disconnected from the second capacitor (C2′) when the switch (Si) is open such that the resonant circuit comprises a first resonance frequency when the switch (S1) is open and a second resonance frequency when the switch (S1) is closed. The second resonance frequency is different from the first resonance frequency. The control circuit (CC) is configured to apply a frequency modulated control signal or a pulse width modulated control signal to the switch (S1) for controlling the switch (S1).

### TECHNICAL FIELD

The present invention relates to an implantable medical device.

### BACKGROUND

Rechargeable implantable medical devices need to have a reliable power supply that can be recharged in an efficient, reliable and safer manner using an external charging device.

Usually, this requires an internal charging circuit capable of controlling the implant's charging voltage and a high-speed communication to communicate information regarding the internal voltage and temperature to the external charging device.

State-of-the-art systems accomplish the recharging function by the use of a high-speed communication link providing temperature and internal voltage of the implant to the external charger where the charging control function resides.

Particularly, current solutions depend exclusively on the external charging device to control the charging voltage internal to the implant. This is done via a closed feedback loop, wherein the implant reports the internal charging voltage to the external charging device. However, such loops comprise a delay which complicates loop stabilization. Furthermore, solutions of this kind cannot respond to unwanted charging fields from unintended sources.

### SUMMARY

Based on the above, the problem to be solved by the present invention is to provide an implantable medical device that can be charged via a charging coil of the medical implant in an efficient manner without generating high voltages within the implantable medical device.

This problem is solved by an implantable medical device having the features of claim 1.

Preferred embodiments of the present invention are stated in the corresponding sub claims and are described below.

According to claim 1, an implantable medical device is disclosed, comprising:
a resonant circuit comprising an inductive charging coil, a first capacitor and a second capacitor, wherein the inductive charging coil is electrically connected to the first capacitor, and
a switch, wherein the inductive charging coil is electrically connected to the second capacitor when the switch is closed and electrically disconnected from the second capacitor when the switch is open such that the resonant circuit comprises a first resonance frequency when the switch is open and a second resonance frequency when the switch is closed, wherein the second resonance frequency is different from the first resonance frequency.

According to the present invention, the implantable medical device comprises a control circuit that is configured to apply a frequency modulated control signal or a pulse width modulated control signal to the switch for controlling the switch.

Advantageously, the present invention provides local control of the charging voltage by detuning the charging coil, thus not wasting power and not generating high voltages within the implantable medical device independent of the source signal.

In other words, the implantable medical device allows controlling the resonance of its charging coil by selectively engaging one or more capacitors to move its resonant frequency away from the frequency of an intended or unintended power source. The purpose of varying the local circuit's resonant frequency is to employ a means of regulating the power taken by the charging coil and regulating the charging voltage as a result.

Particularly, a single capacitor can be switched in periodically for a duration necessary to transfer only the necessary power demanded by the implantable medical device at that time. Particularly, it is also possible that the implantable medical device comprises a plurality of capacitors and switching circuits which could be selected independently. By engaging a combination of these capacitors, the local circuit's resonant frequency could be set to allow coupling to the external charging device's signal at a level appropriate to transfer only power sufficient to the implantable medical device's need at that time.

According to a preferred embodiment, the implantable medical device comprises a circuit that is an electrically floating switch, by means of which a capacitor can be added or removed from the resonant circuit to tune its resonance frequency.

Furthermore, according to a preferred embodiment of the present invention, the switch is opened (turned off) in case the control signal is applied to the switch, and wherein the switch is closed (turned on) in the absence of a control signal. Alternatively, the switch is closed (turned on) in case the control signal is applied to the switch, and wherein the switch is opened (turned off) in the absence of a control signal.

According to a further embodiment of the present invention, the implantable medical device comprises a transformer, wherein the control circuit is connected to a primary winding of the transformer, and wherein a secondary winding of the transformer is connected to the switch.

Preferably, according to an embodiment of the present invention, the transformer comprises an inductor with a core made of non-magnetic material, e.g., an air core. By avoiding magnetic core materials for inductors, the implantable medical device may operate normally in the environment of a magnetic resonance imaging (MRI) machine, because the high RF and magnetic signal levels present in an MRI machine cannot sufficiently couple into the inductor and induce damaging currents.

Furthermore, according to an embodiment, the switch comprises a first terminal (in) and a second terminal (out), wherein the first and the second terminal are connected via a first and a second MOSFET (metal-oxide-semiconductor field-effect transistor), wherein the first and the second MOSFETs are tied together at their sources (that are connected to the local reference node of the circuit, in particular), and wherein the drain of the first MOSFET is connected to the first terminal of the switch, and wherein the drain of the second MOSFET is connected to the second terminal of the switch. When the voltage at the first or second MOSFET's gate is nearly the same as the voltage at the local reference node, the switch is off When the gate voltage is significantly higher (for an N-MOSFET) or lower (for a P-MOSFET) than the voltage at the local reference node, the switch is turned on. For the latter case, significantly can mean 3 to 5 volts, whereby a difference of less than 0.5 volts would not be significant.

According to embodiments of the present invention, N-type MOSFETs or P-type MOSFET can be used for the circuits described. N-type MOSFETs are preferred for switching applications because these have lower on-state resistance for their silicon area.

Further, according to an embodiment, the gates of the first and the second MOSFET are electrically connected to one another.

Furthermore, according to an embodiment, the switch comprises a third MOSFET, wherein the secondary winding of the transformer is connected to the gate of the third MOSFET via a diode, which half-wave rectifies the control signal passed to the gate of the third MOSFET, and wherein the source of the third MOSFET is electrically connected to the sources of the first and of the second MOSFET, and wherein the drain of the third MOSFET is electrically connected to the gates of the first and of the second MOSFET.

Furthermore, according to an embodiment, the switch comprises a holding capacitor connecting the sources of the first and of the second MOSFET to the gates of the first and of the second MOSFET.

Preferably, in an embodiment, the first terminal of the switch is connected via a resistive path comprising a diode and a resistor to the gate of the first MOSFET to allow a current to flow from the first terminal of the switch to the gate of the first MOSFET, and/or wherein the second terminal of the switch is connected via a resistive path comprising a diode and a resistor to the gate of the second MOSFET to allow a current to flow from the second terminal of the switch to the gate of the second MOSFET.

According to yet another embodiment, the switch comprises a diode, a capacitor and a resistor connected to the gate of the third MOSFET, wherein the capacitor and the resistor are connected in parallel.

According to an embodiment, when the control signal is removed, the charge on the capacitor of the combination dissipates through the resistor of the combination and the third MOSFET turns off and charge builds up on said holding capacitor and the first and the second MOSFET enter a low resistance state which is considered as an “on”-state of the switch, i.e., the switch is closed.

According to a further embodiment, applying the control signal to the transformer causes charge to build up on the capacitor of the low pass filter, which then causes the voltage applied to the gate of the third MOSFET to rise to the point where the third MOSFET turns on, discharging the holding capacitor and causing the first and the second MOSFET to enter a high resistance state which is considered as an “off”-state of the switch, i.e., the switch is open.

According to a further embodiment, the implantable medical device is configured to communicate a signal indicative of a temperature of the implantable medical device to an external charging device through a frequency modulation of the control signal.

Furthermore, according to an embodiment, the implantable medical device is configured to communicate a signal indicative of a requested power level to an external charging device through a frequency modulation of the control signal.

Furthermore, according to an embodiment, the implantable medical device is configured to provide one of: neuro stimulation, spinal cord stimulation, deep brain stimulation, vagus nerve stimulation, sacral nerve stimulation.

### DESCRIPTION OF THE DRAWINGS

In the following, embodiments of the present invention as well as further features and advantages shall be explained with reference to the Figures, wherein:

FIG. 1 shows a power receiving circuit of an embodiment of an implantable medical device, wherein the power receiving circuit comprises a switch for connecting a capacitor in parallel to another capacitor of a resonant circuit to tune the resonance frequency of the resonant circuit; and

FIG. 2 shows an embodiment of the switch; and

FIG. 3 shows an exemplary diagram with a pulse width modulated control signal.

### DETAILED DESCRIPTION

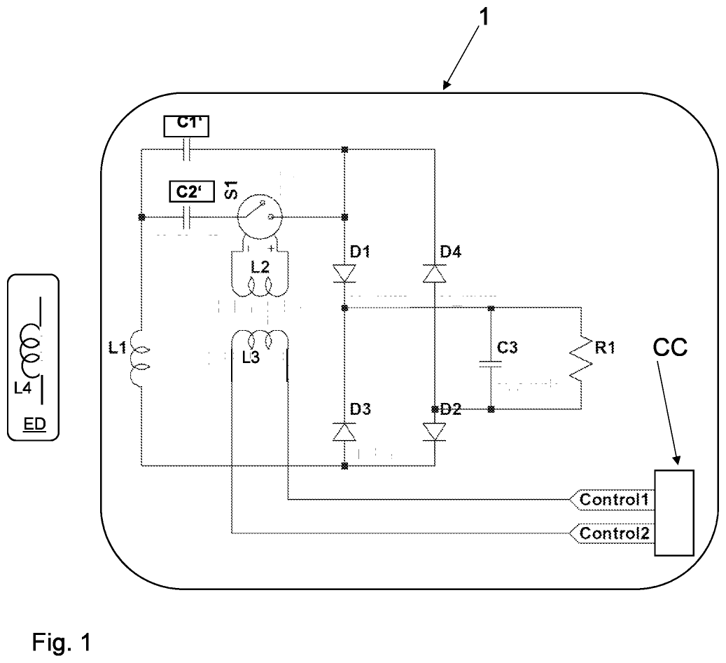

FIG. 1 shows a diagram of an embodiment of an implantable medical device 1 comprising a power receiving circuit which allows power to be coupled into the system through a charging coil L1 (also denoted as pick-up coil). The charging coil L1 is preferably resonated with two capacitors C1 and C2 at the frequency of the intended power transfer signal. A switch S1 is disengaged when a control signal is applied by a control circuit CC across the terminals Control1 and Control2 of the transformer created by L2 and L3. In case the path through C2 and S1 is opened, the circuit resonates at a higher frequency set (predominately) by L1 and C1. In fact, C1 can be removed from the circuit but provides a way to better control the resonance frequency when switch S1 is open. When S1 is open, the circuit is deliberately far less effective at coupling energy into the implantable medical device 1, particularly so at the intended power transfer frequency. Diodes D1 through D4 form a bridge rectifier for converting the received alternating current signal into direct current which can be used to power the implantable medical device 1. The capacitor C3 acts as a filter to smooth the rectified power and resistor R1 represents the electrical load of the implant's circuit and its rechargeable battery. The function of the transformer formed by L2 (secondary winding) and L3 (primary winding) is to provide DC isolation between the alternating current side and the directed current side of the rectifier D1 through D4. According to the embodiment, the resonance frequency of the coil/capacitor combination is shifted higher when one wishes to reduce power transferred.

According to an alternative embodiment, the charging coil L1 and capacitor C1 resonate at the desired frequency for charging when S1 is open. When S1 is closed, L1 resonates with C1 and C2 at a much lower frequency where effective charging is not possible. According to the embodiment, the resonance frequency of the coil/capacitor combination is shifted lower when one wishes to reduce power transferred.

The proposed embodiments provide a circuit for an implantable device which is able to shift the resonant frequency away from the power carrier frequency of the external charger.

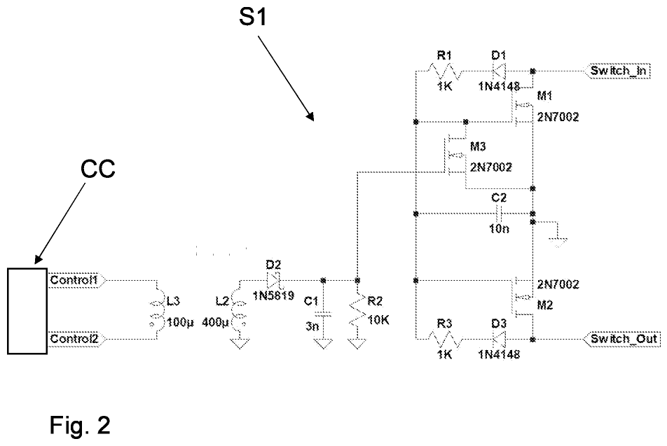

Furthermore, FIG. 2 shows an embodiment of the switch S1 depicted in FIG. 1 together with the transformer L2/L3. The switched terminals of switch S1 are designated as Switch_In and Switch_Out. MOSFETs M1 and M2 commutate said terminals. MOSFETs M1 and M2 are connected such that their sources are tied in common. This common connection point can be considered as the circuit reference. Transistors of this design will provide isolation for current flowing in only one direction, from the drain to the source. Since switch S1 is required to operate in the presence of current alternating in direction, MOSFET M1 provides isolation of current attempting to flow from Switch_In to Switch_Out. MOSFET M2 isolates current attempting to flow in the opposite direction. The drain of the first MOSFET M1 is connected to the switch terminal Switch_In and the drain of the second MOSFET M2 is connected to the switch terminal Switch_Out. The control inputs (gates) of the MOSFETs M1 and M2 are connected together. Diodes D1 and D3 together with resistors R1 and R3, provide a resistive path for power to flow from the switch terminals. Switch_In and Switch_Out to the control terminals of the MOSFETs M1 and M2. Absent the third MOSFET M3, when a sufficiently large AC signal is applied across terminals Switch_In and Switch_Out of switch S1, current flows through diodes D1 and D3, charging the holding capacitor C2. The result is an increasing voltage across the control terminals (gates) to the sources of MOSFETs M1 and M2 which cause MOSFETS M1, M2 to exhibit low resistance from the respective drain to the respective source. In this condition the switch S1 is considered to be “on”. The addition of the third MOSFET M3 provides a means to shunt current away from holding capacitor C2, thus preventing the build-up of voltage on the control terminals (i.e., gates) of the first and the second MOSFET M1, M2. This enforces the “off” condition bringing the resistance between the sources and respective drains for the first and the second MOSFET M1, M2 to a high value. The remaining components, R2, C1, D2, and the transformer comprised of secondary winding L2 and primary winding L3, are configured for controlling the third MOSFET M3. Components capacitor C1 and resistor R2 form a low pass filter to hold the value at the gate of M3 at a steady level for some time duration greater than the cycle time of the signal applied to said transformer. Diode D2 half-wave rectifies the control signal such that only the positive half cycles of the control signal are allowed to pass to control the gate of MOSFET device M3. When said control signal is not present, the charge on filter capacitor C1 dissipates through R2. MOSFET device M3 then turns of charge builds up on holding capacitor C2 and MOSFET devices M1 and M2 enter the low resistance state (i.e., the switch S1 is turned “on”, i.e., is closed). When an alternating current control signal is present at the transformer inputs, this causes charge to build up on filter capacitor C1 then causing the voltage applied to the control input (gate) of the third MOSFET M3 to rise to the point where the third MOSFET M3 turns on, thus discharging holding capacitor C2 and causing the first and second MOSFETs M1, M2 to enter the high resistance state (i.e. the switch S1 is turned “off”, i.e. the switch is open). For system operation purposes, it is important to note that an active control signal turns the switch S1 “off” and that switch S1 is “on” in the absence of a control signal. This allows an implantable medical device 1 with a fully discharged battery to receive power. A switch S1 that depends on an active control signal to engage would not be able to start up in the case where no local power was available to create the control signal.

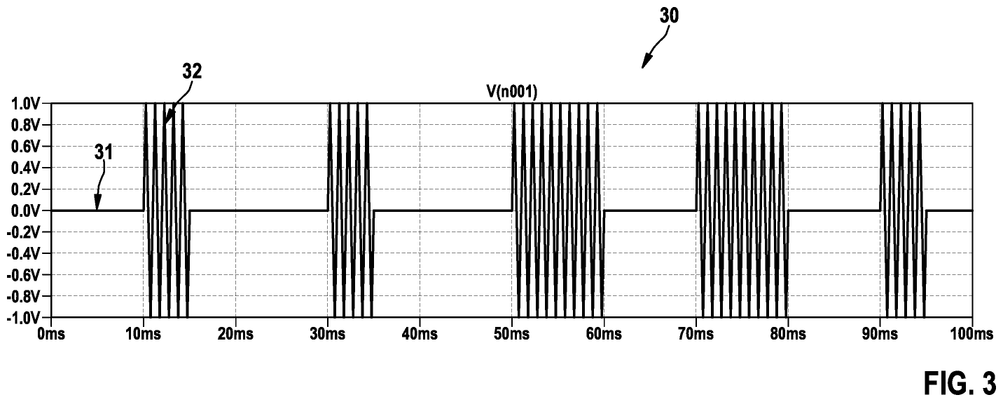

The control signal applied to the control inputs Control1, Control2 must be an alternating current signal of appropriate frequency to couple through the transformer comprised of secondary winding L2 and primary winding L3 effectively. However, the control signal can be on-off modulated in a pulse width fashion where the duty cycle of the modulating pattern sets the voltage applied to the load represented by resistor R1 in FIG. 1. FIG. 3 shows an exemplary diagram with a pulse width modulated control signal having off-phases 31 and on phases 32. In this way the implantable medical device 1 can regulate the internal voltage to an arbitrary voltage that is lower than the maximum voltage possible if the switch S1 were closed 100% of the time. Because the duty cycle of the modulated control signal can be controlled independently of the frequency of the modulating wave form, it is possible to convey information to the external charging device ED from the implantable medical device 1 by detecting the pattern of loading reflected to a transmitting coil L4 of the external charging device ED. Additionally, also the loading duty cycle may be ascertained. The frequency modulated data can convey useful information to the external charging device ED such as device temperature or the desired charger transmitting power setting. As an alternative, the external charging device ED itself can make adjustments in its power setting directly based on the observed duty cycle. Particularly, the frequency modulation can be continuous to infer analog information or discrete to infer digital values. One example use of such communication would be for the implantable medical device 1 to control the transmitter power level of the external charging device ED as a means to regulate the temperature of the implantable medical device 1. Ideally, for minimal heating, the “on” state duty cycle should be high as the enclosure and battery case are inductively heating proportionally to the charger transmitting power independently of the state of switch S1. Therefore, the implantable medical device 1 would direct the charger transmitting power level to, for example, allow sufficient power transfer at 80 to 90 percent switch S1 on-time duty cycle. This would allow some margin for power consumption variation while limiting can and battery case losses to only 10 to 20 percent above the minimally necessary levels.

Specific preferred embodiments of the implantable medical device 1 according to the present invention are:
The implant regulates its internal voltage, and the external charging device ED sets the power level based on the observed duty cycle of the load on the transmitting coil L4 of the external charging device ED. The implantable medical device 1 can communicate temperature information via frequency modulation and the external charging device ED can respond by adjusting its transmit power level to keep the temperature of the implantable medical device 1 in an acceptable range.
The implantable medical device 1 regulates its internal voltage. Further, it communicates a requested power level to the external charging device ED through frequency modulation as described above. In this case the implant is in full control of the charging loop with the entire controller portion of the system being within the implantable medical device 1. The external charging device ED simply responds to the requests of the implantable medical device 1. In this configuration the output signal to the external charging device ED could be based on a combination of power demand and temperature of the implantable medical device 1 and as such the implantable medical device 1 would be in control of the tradeoff between charging time and temperature rise.

The present invention offers the advantage of a more predictable power transfer behavior which can:
improve the predictability of battery charging times,
reduce voltage stress on circuit components allowing smaller components to be used,
reduce power received from untended sources reducing or eliminating failures induced by excessive charging signals that could come from e.g., theft detection systems or misuse of other chargers, such as charging devices using a Xi wireless charging protocol.

Furthermore, due to the fact that the power control for charging can be moved to the implantable medical device, the safety class of the external charging device can be lower.

According to an embodiment of the present invention, all components of the switch according to the invention, eventually except for the control transformer, can be implemented as a single integrated circuit. Furthermore, according to an embodiment, a Hall effect sensor or a GMR sensor can be implemented on the same circuit to replace the control transformer.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

1. An implantable medical device (1), comprising
a resonant circuit comprising an inductive charging coil (L1), a first capacitor and (C1′) a second capacitor (C2′), wherein the inductive charging coil (L1) is electrically connected to the first capacitor (C1′) of the resonant circuit;
a switch (S1), wherein the inductive charging coil (L1) is electrically connected to the second capacitor (C2′) of the resonant circuit when the switch (S1) is closed and electrically disconnected from the second capacitor (C2′) when the switch (S1) is open such that the resonant circuit comprises a first resonance frequency when the switch (S1) is open and a second resonance frequency when the switch (S1) is closed, wherein the second resonance frequency is different from the first resonance frequency; and
a control circuit (CC) that is configured to apply a frequency modulated control signal or a pulse width modulated control signal to the switch (S1) for controlling the switch (S1).

2. The implantable medical device according to claim 1, wherein the switch (S1) is configured to open in response to the control signal being applied to the switch (S1) and wherein the switch (S1) is configured to close in the absence of a control signal.

3. The implantable medical device according to claim 1, wherein the implantable medical device (1) comprises a transformer (L3, L2), wherein the control circuit (CC) is connected to a primary winding (L3) of the transformer, and wherein a secondary winding (L2) of the transformer is connected to the switch (S1).

4. The implantable medical device according to claim 1, wherein the switch (S1) comprises a first terminal (Switch_In) and a second terminal (Switch_out), wherein the first and the second terminal are connected via a first and a second MOSFET (M1, M2), wherein the first and the second MOSFET (M1, M2) are tied together at their sources, and wherein the drain of the first MOSFET (M1) is connected to the first terminal (Switch_In) of the switch (S1), and wherein the drain of the second MOSFET (M2) is connected to the second terminal (Switch_out) of the switch (S1).

5. The implantable medical device according to claim 4, wherein the gates of the first and of the second MOSFET (M1, M2) are electrically connected to one another.

6. The implantable medical device according to claim 4, wherein the implantable medical device (1) comprises a transformer (L3, L2), wherein the control circuit (CC) is connected to a primary winding (L3) of the transformer, and wherein a secondary winding (L2) of the transformer is connected to the switch (S1), wherein the switch (S1) comprises a third MOSFET (M3), wherein the secondary winding (L2) of the transformer is connected to the gate of the third MOSFET (M3) via a diode (D2) of the switch (S1), which half-wave rectifies the control signal passed to the gate of the third MOSFET (M3), and wherein the source of the third MOSFET (M3) is electrically connected to the sources of the first and of the second MOSFET (M1, M2), and wherein the drain of the third MOSFET (M3) is electrically connected to the gates of the first and of the second MOSFET (M1, M2).

7. The implantable medical device according to claim 4, wherein the switch (S1) comprises a holding capacitor (C2) connecting the sources of the first and of the second MOSFET (M1, M2) to the gates of the first and of the second MOSFET (M1, M2).

8. The implantable medical device according to claim 4, wherein the first terminal (Switch_In) of the switch (S1) is connected via a resistive path comprising a diode (D1) and a resistor (R1) to the gate of the first MOSFET (M1) to allow a current to flow from the first terminal (Switch_In) of the switch (S1) to the gate of the first MOSFET (M1), and/or wherein the second terminal (Switch_out) of the switch (S1) is connected via a resistive path comprising a diode (D3) and a resistor (R3) to the gate of the second MOSFET (M2) to allow a current to flow from the second terminal (Switch_out) of the switch (S1) to the gate of the second MOSFET (M2).

9. The implantable medical device according to claim 6, wherein the switch (S1) comprises a diode, a capacitor and a resistor connected to the gate of the third MOSFET (M3), wherein the capacitor (C1) and a resistor (R2) are connected in parallel.

10. The implantable medical device according to claim 9, wherein the switch (S1) comprises a holding capacitor (C2) connecting the sources of the first and of the second MOSFET (M1, M2) to the gates of the first and of the second MOSFET (M1, M2), wherein the resonant circuit, the switch (S1), and the control circuit are configured such that, when the control signal is removed, the charge on the capacitor (C1) dissipates through the resistor (R2) and the third MOSFET (M3) turns off and charge builds up on the holding capacitor (C2) and the first and the second MOSFET transistors (M1, M2) enter a low resistance state such that the switch is closed.

11. The implantable medical device according to claim 9, wherein the switch (S1) comprises a holding capacitor (C2) connecting the sources of the first and of the second MOSFET (M1, M2) to the gates of the first and of the second MOSFET (M1, M2), wherein the resonant circuit, the switch (S1), and the control circuit are configured such that, applying the control signal to the transformer (L2, L3) causes charge to build up on the capacitor (C1) then causing the voltage applied to the gate of the third MOSFET transistor (M3) to rise to the point where the third MOSFET transistor (M3) turns on, discharging the holding capacitor (C2) and causing the first and the second MOSFET transistor (M1, M2) to enter a high resistance state such that the switch is opened.

12. The implantable medical device according to claim 1, wherein the implantable medical device (1) is configured to communicate a signal indicative of a temperature of the implantable medical device (1) to an external charging device (ED) through a frequency modulation of the control signal.

13. The implantable medical device according to claim 1, wherein the implantable medical device (1) is configured to communicate a signal indicative of a requested power level to an external charging device (ED) through a frequency modulation of the control signal.

14. The implantable medical device according to claim 1, wherein the implantable medical device (1) is configured to provide one of: neuro stimulation, spinal cord stimulation, deep brain stimulation, vagus nerve stimulation, or sacral nerve stimulation.

15. The implantable medical device according to claim 3, wherein the transformer (L3, L2) comprises an inductor with a core, wherein the core is made of non-magnetic material.
